(19) United States
(12) Patent Application Publication
(10) Pub. No.: US 2007/0038311 A1
(43) Pub. Date: Feb. 15, 2007

(54) System and method for improving the functionality of prostheses

(75) Inventors: Todd A. Kuiken, Oak Park, IL (US); Richard Weir, Evanston, IL (US); Jon Sensinger, Chicago, IL (US)

Correspondence Address: GARDNER CARTON & DOUGLAS LLP;ATTN: PATENT DOCKET DEPT., 191 N. WACKER DRIVE, SUITE 3700, CHICAGO, IL, 60606, US

(73) Assignee: Rehabilitation Institute of Chicago, Chicago, IL (US)

(21) Appl. No.: 11/503,472

(22) Filed: Aug. 11, 2006

Related U.S. Application Data
(60) Provisional application No. 60/707,481, filed on Aug. 11, 2005.

(51) Int. Cl. A61F 2/70

(52) U.S. Cl. 623/24

(57) ABSTRACT

A system and method for improving the functionality of a prosthesis used by an amputee in which a portion of the user's skin is reinnervated with nerves that formerly provided sensory feedback from the lost limb, providing a haptic indication from the prosthesis, and providing a corresponding haptic effect at the surface of the reinnervated skin. The reinnvervated skin provides transfer sensation that supplies the user with the psychological reassurance of sensing touch in the prosthesis while helping to meet the practical needs of enabling goal confirmation and the application and sensing of graded pressure in the prosthesis.

### CROSS-REFERENCE TO RELATED APPLICATION

This application claims the benefit of U.S. Provisional Application No. 60/707,481, filed Aug. 11, 2005, and incorporated by reference in its entirety.

### FIELD OF THE INVENTION

This invention pertains to the field of prosthetics. More particularly, this invention relates to a system and method for providing haptic feedback from external prostheses to enhance the functionality of such devices.

### BACKGROUND OF THE INVENTION

Improving the functionality of prostheses, such as artificial upper and lower limb prostheses, is a considerable challenge, especially for high-level amputations where the disability presented by the amputations is the greatest. In the United States during the period from 1988 to 1996 more than 100,000 people lost at least a part of an upper limb (thumb, finger, hand, wrist or transradial, elbow disarticulation, transhumeral, shoulder disarticulation or forequarter amputations) mostly as a result of trauma, dysvascularization or cancer. Lower limb amputations are even more ubiquitous with over 50,000 cases per year in the United States alone. While prosthetic devices can help people perform some daily activities, many upper and lower limb amputees still find that their prostheses have unsatisfactory functionality and do not use them. As a result, many prosthetic users choose not to wear a prosthesis at all.

Conventional prosthetic devices, including body powered and motorized hooks, hands, wrists, elbows, knees, feet, etc. are nevertheless used by many amputees in performing activities of daily living. Such prosthetic devices do not provide the full functionality of a natural limb. For example, conventional prostheses do not allow a user to feel the force or pressure applied by or to the prosthesis. As a result, conventional upper and lower limb prostheses do not give the user the psychological reassurance of sensing touch in the prostheses. Conventional hand prostheses also do not meet the practical needs of allowing a user to sense, without visually observing the prostheses, whether they are gripping an item, let alone whether they are holding it loosely or tightly. Thus, items held in a conventional prosthesis may be dropped because they are not held securely or they may be crushed due to the application of excessive gripping force. Conventional foot prostheses do not allow the user to sense pressure on the foot prostheses as the user walks. This adds to the difficulty of learning to use and then using such devices.

Currently, most powered artificial limbs are controlled using myoelectric signals from an antagonist pair of muscles in the amputated limb. This allows only a single form of motion to be performed at a time and is therefore unduly cumbersome. Furthermore, such devices currently provide no haptic feedback.

Although a limb is lost with an amputation, the control signals to the limb remain in the residual peripheral nerves. In recently developed upper limb prostheses, these control signals are tapped into, using nerve transfers that greatly improve the control and function of the prostheses. See Kuiken T A, Rymer W Z, Childress D S (1995), “The Hyper-reinnervation of Rat Skeletal Muscle,” Brain Res 676, 113-123; Kuiken T A, Stoykov, Popovic M, Lowery M and Taflove A (2001), “Finite Element Modeling of Electromagnetic Signal Propagation in a Phantom Arm,” IEEE Trans Neural Sys and Rehab Engr 9(4), 345-354; Kuiken T A T A, Lowery M M and Stoykov N S, “The Effect of Subcutaneous Fat on Myoelectric Signal Amplitude and Cross-Talk,” Prosthetics and Orthotics International 27, pp 48-54, 2003; Kuiken T A, Dumanian G A, Lipschuzt R D, Miller L A and Stubblefield K A, “Targeted Muscle Reinnervation for Improved Myoelectric Prosthesis Control,” Prosthetics and Orthotics International, 28(3) pp. 245-253, December 2004, the entirety of which are incorporated by reference. It has thus been demonstrated that it is possible to control prostheses using such nerve transfers. This involves denervating expendable regions of muscle in or near an amputated limb and transferring the residual peripheral nerve endings to these muscles. The nerves reinnervate these muscles. Then, the surface electromyograms (EMGs) from the nerve-muscle transfers are used as additional myoelectric control signals for an externally powered prosthesis. While these new prosthetic control techniques represent a very significant advance in the art, even such a highly articulated limb controlled by surface EMGs from the nerve transfers would be substantially improved if haptic feedback could be provided.

With the nerve transfer technique discussed above, the amputee's residual nerves are transferred onto “foreign” regions of muscle and cross-reinnervate these muscles. Using such nerve transfers for amputees takes advantage of the nerves' inherent motor programming so that the nerves simultaneously control physiologically appropriate functions in the prosthesis. The control of the artificial limb has been demonstrated successfully in several patients. They report targeted reinnervation control to be quicker and to have a more natural feel than with their prior conventional myoelectric prostheses. This reduces the conscious effort required by the amputee, making the prosthesis easier to use and more functional.

The nerve transfer control technique discussed above may be used with existing myoelectric technologies. Powered elbows, wrists and terminal devices are commercially available with circuitry allowing up to seven analog inputs (e.g. myoelectric signals) and four on/off input signals that provide the control of up to five motors. The nerve transfer technique enables better control of such complex prosthetic devices but still lacks the haptic feedback necessary for optimal human control.

For the nerve transfer control technique to be successful in amputees, multiple nerves must consistently reinnervate separate regions of muscle. In the past, muscle recovery after nerve transection has been inconsistent and often unsatisfactory. However, in order to address this issue, optimally large nerves containing many times the normal number of motoneurons are grafted onto the muscles thus “hyper-reinnervating” the muscles. Hyper-reinnervating muscle (grafting an excessive number of motoneurons onto a muscle) increases the likelihood that any given muscle fiber will be reinnervated and this improves muscle recovery. A related issue is containment of the reinnervation field. With the nerve transfer technique multiple nerves will be grafted onto different regions of a muscle, each with nerve reinnervating only the intended muscle region. Also, cross-talk is prevented from interfering with prosthesis operation by setting a threshold above background noise and the cross-talk from nearby muscles. The amputee must generate an EMG signal greater than the threshold to operate the prosthesis.

It is therefore an objective of this invention to provide a system and method for providing haptic feedback to an amputee using a prosthesis.

It is another object of the invention to enable a user of a prostheses to regain a sense of touch.

It is a further object of the present invention to provide an amputee using a prosthesis with a sense of force/pressure, temperature, vibration, texture, or sharp/dull edges at various locations on the prosthesis.

It is yet another object of the present invention to reinnervate an area of skin on an amputee's body with nerves that formerly provided sensory feedback from the part of a lost limb replicated by a prosthesis and to supply sensory input from the prosthesis to the reinnervated area.

It is a still further object of the present invention to provide an amputee wishing to use a prosthesis to grip an item with haptic feedback allowing the amputee to sense, control, and adjust the tightness or looseness of the grip.

Yet another object of the invention is to provide lower limb amputees with prostheses that enable the amputee to sense pressure on the prosthesis as he walks.

Still another object of the present invention is the provide an enhancement of systems using nerve transfers as control signals for powered prostheses in which the amputee is also supplied with haptic input from selected areas of the prostheses to areas of reinnervated skin.

These and other objects and advantages are achieved in the practice of the present invention as described below.

### SUMMARY OF THE INVENTION

The present invention may be used with prostheses for any amputation that would benefit from haptic feedback. For example, it may be used with prostheses for transcarpal and higher upper limb amputations and partial foot and higher lower limb amputations. The sensory information may be any information that is available to nerve endings on the skin including force or pressure, texture, temperature, vibration and sharp/dull and edge sensations so long as appropriate corresponding nerves from the amputated limb can be relocated to a reinnervated skin area and accessed in that area.

The present invention relies on sensory nerve transfers. Motor nerve transfers as described above in the Background of the Invention are used to gain additional motor commands for operating a prosthesis by surgically moving residual limb nerves to muscles. This invention applies a similar concept to skin. Thus, nerve to an area is cut to denervate the skin and new sensory nerve fibers grow into the skin. When this skin is then touched, the person feels like they are being touched in the area that used to innervated by the transferred nerve. For example, the residual arm nerves of a person with a shoulder disarticulation amputation have been transferred to different sections of the chest muscles. The nerves to the skin over these nerve transfers were also cut. Then the sensation nerves to the hand reinnervated the chest skin. When this chest skin is touched, the person feels the touch in their missing hand. They feel light touch, graded pressure, hot, cold, sharp and dull—all as if it were in there missing hand. This reinnervated skin is thus serving as a mechanism to provide sensation to a missing body part. This is referred to in the context of the present invention as “transfer sensation.”

In the practice of the present invention, haptic feedback is provided from a prosthesis to these nerves that formerly served the missing natural limb to enhance the function and control of the prosthesis. According to one embodiment of the invention, a prosthesis is equipped with appropriate sensor(s) and the person using the prosthesis is fitted with one or more transducers that can selectively produce sensory feedback (a “sensory condition”) over one or more locations on the user's skin that have been reinnervated with nerves that formerly provided sensation to the amputated limb (e.g., hand or foot). Now touching this skin provides transfer sensation. When this skin is touched, it feels like the missing hand or foot is being touched. Sensors on the prosthesis will measure the interaction of the prosthesis and an object (e.g., pressure, texture, temperature) and an actuator over the reinnervated skin will apply an appropriate stimulation so the user “feels” what the prosthesis is touching as if it were their own hand or foot.

The sensor, transducer, and coupling therebetween and to the reinnervated skin (using nerves in the reinnervated skin that formerly provided sensation from the part of the lost limb replicated by the prosthesis) together comprise the haptic interface of the invention. For example, the interface may include a pressure sensor located at a tip of a motor-driven movable finger or toe of a prosthesis, where the pressure sensor is capable of detecting and producing an indication of the level of pressure applied to the finger or toe. The person using the prosthetic will be fitted with an actuator that selectively applies a force to the skin reinnervated with the nerves that formerly controlled the tip of the user's finger or the pad of the toe. The actuator is preferably of a type that can exert varying degrees of force on the appropriate location(s) of the reinnervated area corresponding to the pressure indication from the pressure sensor. Preferably the force applied by the actuator will be scaled down since the reinnervated skin typically will be substantially more sensitive or tender than the corresponding area of the natural limb where the pressure would have been sensed but for the events resulting in the amputation and placement of the prosthesis. Also, the pressure is typically better cushioned and more distributed in the natural limb area. The scaling may be linear or non-linear. For example, the pressure may be unscaled or scaled up at lower pressures and scaled down at higher pressures. The scaling where it occurs may be, for example, linear initially and then exponential.

The interface includes the sensor in the prosthetic limb and the actuator over the reinnervated skin and may also include a controller that receives a signal or other indication from the prosthesis sensor corresponding to the level of pressure, texture or temperature detected whereby the actuator exerts either a corresponding force, vibration or temperature or a scaled value on the appropriate location(s) of the skin reinnervated with nerves that formerly controlled the tip of the user's finger. As a result, the amputee senses the force, vibration or temperature through the nerve formerly associated with the lost natural limb in a way corresponding to the way it had been sensed in the now absent natural limb. This feedback makes the prosthetic feel more natural and satisfying and it helps the amputee to control the pressure applied by e.g., a hand prosthesis, or to operate e.g., a leg/foot prosthesis with the benefit of sensing pressure on the toes, ball and heel of the foot. For example, by providing such feedback from portions of some or all of the fingers of a prosthetic hand, the user will have a transfer sensation corresponding to the amount of gripping pressure a prosthetic hand is exerting on an object.

Various embodiments of the present invention provide apparatus and methods for providing an amputee with haptic sensations from a prosthetic device. Thus, the prosthetic sensors can be pressure sensors, vibration sensors, edge detectors or temperature sensors. Non-limiting examples of pressure sensors that may be used include strain gauge sensors and force sensitive resistors (which e.g., may be embedded in the fingertips of the prosthesis). A thermistor may be used as a temperature sensor. An accelerometer may be used to sense vibration.

The prosthetic actuators may be any device capable of responding to the sensor (directly or through a controller) to produce a corresponding or scaled output at the appropriate location(s) of the reinnervated skin area. The actuators will be adapted to provide the appropriate form of sensory feedback to the reinnervated tissue location, e.g., a load actuator will produce a level of force corresponding (scaled down if needed) to the pressure sensed by a pressure sensor and a temperature actuator will heat or cool the appropriate location(s) of the reinnervated skin area to a degree corresponding (or scaled down but proportional) to a temperature detected by a temperature sensor. It is preferred that the actuators include means for monitoring the level of pressure, temperature, etc. applied to insure that the intended level is reached. This monitoring function may be performed by the controller.

Examples of pressure actuators include the devices of FIGS. 3-7 described below in the Detailed Description of the Invention as well as the pressure actuator of Example 1 appearing thereafter. Also, an elastic actuator such as described in U.S. Pat. No. 5,650,704 may be used as a pressure transducer and an electroactive polymer actuator such as described in U.S. Pat. No. 6,809,462 may also be used. These patents are incorporated by reference in their entirety. It is also possible to employ a linear solenoid having a plunger with an axial stroke as a pressure actuator. Peltier devices may be used as temperature actuators supplying heating or cooling to the appropriate reinnervated locations.

Force sensors, pressure sensors or accelerometers may also be used to sense texture. Thus when an amputee passes a portion of the prosthesis fitted with a pressure sensor over a surface, the rapid variation in pressure as the pressure sensor moves over the surface will be reflected at the reinnervated skin site by the pressure actuator so long as the pressure sensor and actuator are adapted to respond at or near the same frequency as the pressure changes encountered. Texture could also be transmitted by a multiplicity of independent pressure sensors that send pressure data to a multiplicity of different pressure sensitive nerve endings at the reinnervated skin site.

Sharp/dull or edge surface features may be transmitted to the reinnervated skin site by taking pressure readings with sensors with a series of closely packed pins or edges that are oriented generally perpendicular to the prosthesis in the area where the sharp/dull or edge features are to be sensed. These sensors will work with actuators that will produce a corresponding sharp/dull or edge sensation at the reinnervated skin. Other sharp/dull or edge sensors and actuators which are currently known or developed in the future could also be used and are incorporated by reference.

The method of the invention may be performed as follows:
1. Reinnervate an Accessible Skin Area of an Amputee with Nerve Transfers; Re-route Nerves Formerly Associated with a Lost Limb.

To date, such reinnervation has been accomplished as an adjunct to procedures for reinnervating an area of muscle. For example, nerves from an amputated arm have been transferred to chest muscles or to nerves in the chest so that nerves that formerly controlled a portion of the arm (e.g., wrist, fingers, biceps, etc.) instead are used to produce movement of a muscle in the chest. When the person thinks “close hand” or “curl bicep” for example, the transferred nerves cause specific chest muscles to contract to trigger a detection device. Of course, areas of the body other than the chest could be reinnervated in this way so long as there is suitable healthy remaining tissue for a particular patient and the particular limb that has been lost. A prosthetic device can be adapted to interface with the reinnervated tissue area so that when the wearer “thinks” to perform a particular action of a lost limb (e.g., contract biceps), a portion of the reinnervated tissue moves instead, triggering a detection device that, through a controller, actuates motion of the prosthetic device that corresponds to the desired motion (e.g., retract bicep).

In addition to the motor reinnervation of the muscle, sensory cross-reinnervation can be made to occur in the skin of the chest wall. When, following such muscle reinnervation, the chest wall is touched lightly in different places the patient will experience a transfer sensation of a light touch to different parts of his hand and arm. This sensory transfer takes place over the region where the key median and ulnar nerves along with the sensory nerves in the nerve bundle in the arm are anastomosed such as described in Example 1 below. The same would, of course, apply to nerves in the leg. While the present invention may be most useful to individuals who have undergone such targeted nerve-to-muscle reinnervation, it is also beneficial to individuals who undergo only denervation and reinnervation of an accessible skin area. Thus, targeted sensory reinnervation to produce transfer sensation is achieved by denervating sections of remaining skin in an amputee after which the sensation nerves of the hand or feet are guided to reinnervate this skin. Then, when the target skin is touched, warmed, etc., it will feel like the hand or foot is being touched, warmed, etc. The amputee will have near normal light touch levels, graded pressure, sharp/dull sensation, and thermal sensation—all in the missing limb.
2. Fit the Amputee with a Prosthesis.

The prosthetic prosthesis preferably will be motor powered and will be operable using EMG from nerve-muscle transfers as discussed above. Other types of prosthetic devices may however be used.

Appropriate pressure or temperature sensors will be located on or in the surface of appropriate portions of the prostheses. For example, in an arm/hand prosthesis the important areas are the tips of the fingers and the palm area. In a foot prosthesis, the important areas are the ball and heel and the pads of the toes.
3. Identify the Appropriate Haptic Locations in the Reinnervated Skin.

For pressure sensing, a probe may be pressed lightly against portions of the reinnervated skin and the patient asked to indicate when he feels pressure and where in the former natural limb the pressure seems to originate. These locations will be marked for later use with pressure actuators.

For temperature sensing, a heated or cooled probe will be touched against portions of the reinnervated skin and the patient asked to indicate when he feels hot or cold and where in the former natural limb the hot or cool sensations seem to originate. These locations will be marked for later use with temperature actuators.
4. Position the Actuators.

The pressure and temperature actuators will be located preferably at the locations identified in the prior step so that the pressure and temperature will be sensed as coming from prosthetic locations corresponding to former natural limb locations.
5. Provide Appropriate Coupling and Controller.

The sensors and corresponding actuators are either hardwired or they are wirelessly interconnected by using radio frequency (RF) transmitters and receivers. Also, a controller is interposed between the sensor(s) and actuator(s) to provide the power and electronics necessary for powering those components, for providing scaling and amplification as required, for monitoring and maintaining the desired pressure or temperature at the actuator, etc.

Example 1 below focuses on force or pressure sensing which is key to grasping. That is, appropriate force or pressure feedback gives the amputee goal confirmation in his use of a grasping prosthetic and also makes it possible for him to apply graded pressure with the prosthesis.

### BRIEF DESCRIPTION OF THE DRAWINGS

FIG. 1 is a top-level diagrammatic representation of an embodiment of the present invention;

FIG. 2 is a diagram illustrating the placement of components in an embodiment of the present invention in a person with a shoulder disarticulation amputation;

FIG. 3 is a top-level diagrammatic representation of an embodiment of the actuator of FIG. 1;

FIGS. 4-7 are diagrams illustrating other embodiments of the actuator of FIG. 1;

FIGS. 8 and 9 are representations of the upper torso of a shoulder disarticulation amputee showing respectively nerve transfers of the chest area and transfer sensation locations after denervation of the area; and

FIG. 10 is a representation of a series elastic actuator that can be used in the practice of the invention.

### DETAILED DESCRIPTION OF A PREFERRED EMBODIMENT OF THE INVENTION

The following example further illustrates the invention but should not be construed as in any way limiting its scope.

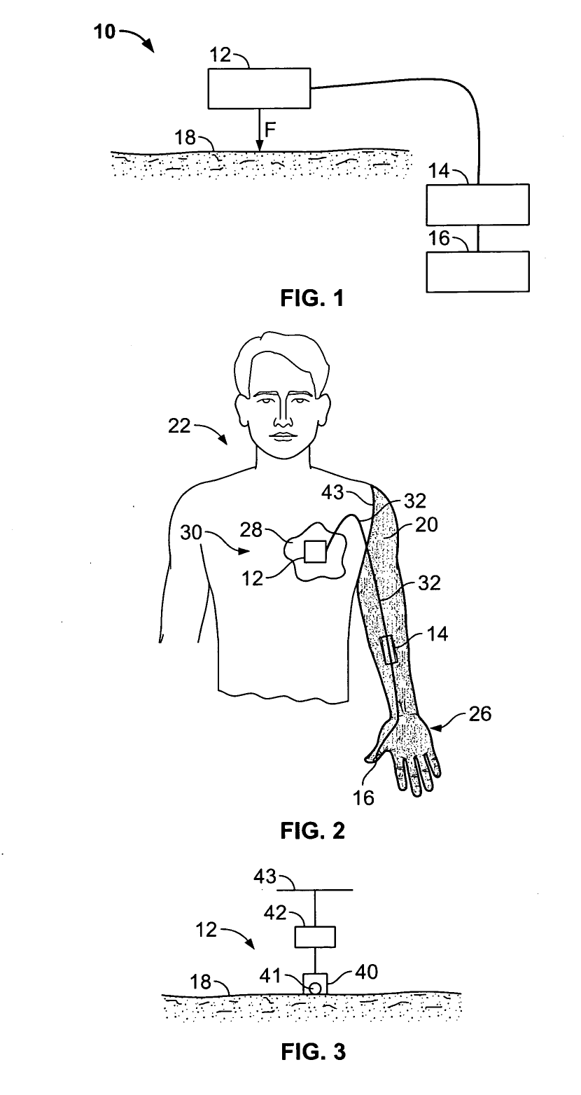

In one embodiment of the present invention, a haptic interface 10 is shown diagramically in FIG. 1, including an actuator 12, a controller 14 and a pressure sensor 16. Controller 14 receives signals from pressure sensor 16 and causes actuator 12 to apply varying pressure to skin surface 18 corresponding to the varying pressure sensed by pressure sensor 16.

Pressure sensor 16 may be mounted on a motor controlled portion of a prosthetic. The controller is coupled to a actuator positioned adjacent the surface of reinnervated skin containing nerves formerly associated with the portion of a user's lost limb now corresponding to the portion of the prosthetic equipped with the pressure sensor. The controller is coupled to the pressure sensor positioned on the prosthetic device and to an actuator that is capable of applying pressure to the reinnervated skin. The pressure sensor thus sends a signal indicating a pressure magnitude to the controller. In response, the controller actuates the actuator to apply a pressure to an area of skin corresponding to (or scaled relative to) the pressure magnitude detected by the sensor. As a result, the pressure applied to the skin stimulates nerves in the skin to transmit sensations to the brain, thereby providing a pressure transfer sensation of a recognizable magnitude and location.

This is illustrated diagrammatically in FIGS. 2 & 3 which show a mechanical prosthetic upper limb 20 worn by an individual 22. Controller 14 is coupled to pressure sensor 16 on thumb pad of a mechanical prosthetic hand 26 and to an actuator 12 that applies pressure to an area of skin 28 on the individual's chest 30 by way of any device or structure that physically contacts and applies pressure to the skin, referred to hereinafter as “tactor 40.” (For example, a plunger in a solenoid type actuator.) This area of skin on the individual's chest has been reinnervated with the nerves that were previously coupled to the individual's natural hand. Controller 14 is coupled to the sensor on thumb pad and to the actuator via conductive wiring 32. Alternatively, other forms of signal transmission could be used, such as wireless RF transmission. Means 41 is also provided for measuring the level of pressure applied by actuator 12 to ensure that the proper level is reached and maintained in accordance with the signal from the sensor. The output of measuring means 41 is supplied to the controller which includes the appropriate electronics to monitor the actual pressure applied by the tactor and make changes or produce error signals as appropriate.

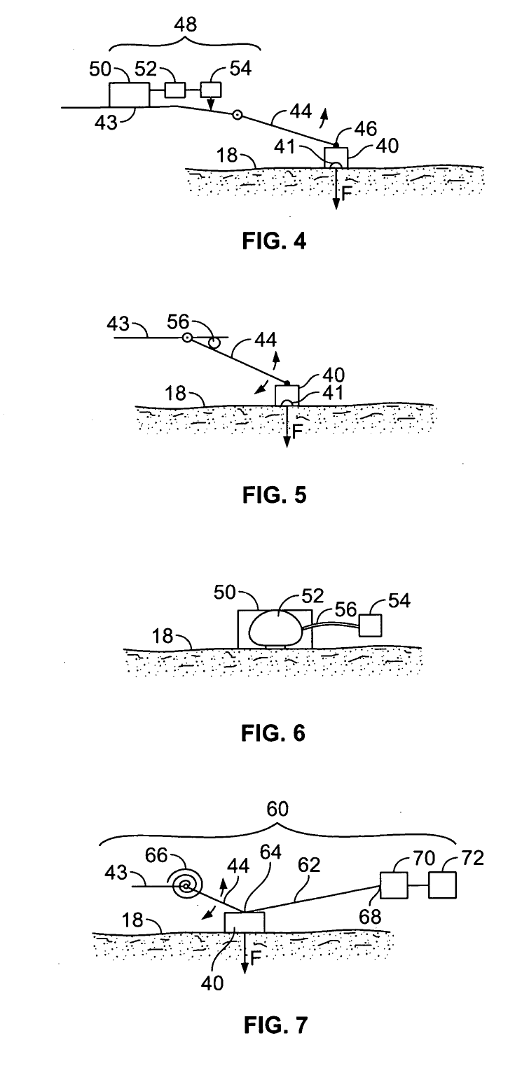

As shown diagrammatically in FIG. 3, actuator 12 can be any available device for applying pressure in response to an electrical signal. For example, actuator 12 may comprise a tactor 40 for contacting reinnervated skin and a driver 42 braced against the prosthetic joint socket 43. When actuated, the tactor 40 applies a force “F” (shown in FIGS. 4, 5 and 7) to the reinnervated skin. The tactor 40 may be operated, for example, by a rack and pinion drive or by a solenoid.

As explained above, in another embodiment illustrated in FIG. 4, actuator 12 includes a pivotal arm 44 having a tactor 40 at its distal tip 46 contacting reinnervated skin surface 18 and a drive 48 to pivot the arm with a desired force. The drive acting on the pivotal arm may comprise, for example, a motor 50, a gear box 52 and a cam drive 54 shown diagrammatically in block form braced against prosthetic joint socket 43.

Alternatively, as shown in FIG. 5, motor 50 of FIG. 4 may be replaced by a pneumatic bladder 56 or a piezoelectric actuator (not shown) braced against prosthetic joint socket 43. FIG. 6 shows another pneumatic bladder driven transducer including a container 50 attached to the reinnvervated skin with an opening opposite a portion of the reinnvervated skin. When bladder 52 is inflated, it will press against the reinnervated skin surface 18. An inflator 54 and hose 56 are provided to inflate and deflate the bladder 52 in response to varying pressure signals from the actuator on the prosthesis. Since the area of the contact between the bladder 52 and the reinnervated skin surface 18 will be known and the air pressure supplied to the bladder 52 will also be known, the pressure applied to the reinnervated skin can be easily determined and adjusted as appropriate (e.g., by the controller) to correspond to the sensor pressure sensed by the actuator on the prosthesis. Thus, bladder 52 offers another approach to producing the desired pressure at the reinnervated skin surface 18.

In FIG. 7, a pressure actuator is shown including a drive 60 having a cable 62 with a first cable end 64 operably linked to arm 44 attached to tactor 40 which may be spring-loaded with spring 66 as shown. The opposite cable end 68 is actuated by a motor 72 and a cable reel box 70. The spring 66 applies a generally constant pressure against the plunger which is opposed by the cable as it is drawn up by operating motor 72 to wind the cable (preferably within a protective housing (not shown)) 62 onto a reel of cable reel box 70. This spring and cable actuator design make it possible to locate the drive remotely from the reinnervated skin to facilitate a low profile design that occupies less space over the critical area of reinnervated skin.

As explained above, in alternative embodiments of the invention, sensors for determining temperature may also be provided to the prosthetic device. The controller processes signals from these additional sensors for operating appropriate temperature actuator. Also, a single sensory device may detect multiple sensory conditions, such as pressure and temperature and a single actuator may impart multimodal stimulus to the reinnervated skin. For example, part 40 in these diagrams may include instead of a tactor a Peltier device capable of heating and cooling. Thus a single actuator may provide the transfer sensations of pressure (low frequency force), texture (an additional high frequency vibration) and temperature—all in the same location with the same device.

The following examples describe embodiments of the present invention and should not be construed as limiting its scope in any way.

### EXAMPLES

1. This example describes targeted reinnervation to transfer nerves from a lost limb to denervated pectoralis muscle, achieving sensation of the lost limb on the chest of a subject. To evaluate this sensation as a potential for feedback in accordance with the invention, a high compliance/low inertia series elastic actuator could be used to apply force to the skin surface over the pectoralis muscle. The subject will have good force resolution when an external force is applied using an instrumented terminal device.

### Setup

Nerve Rewiring

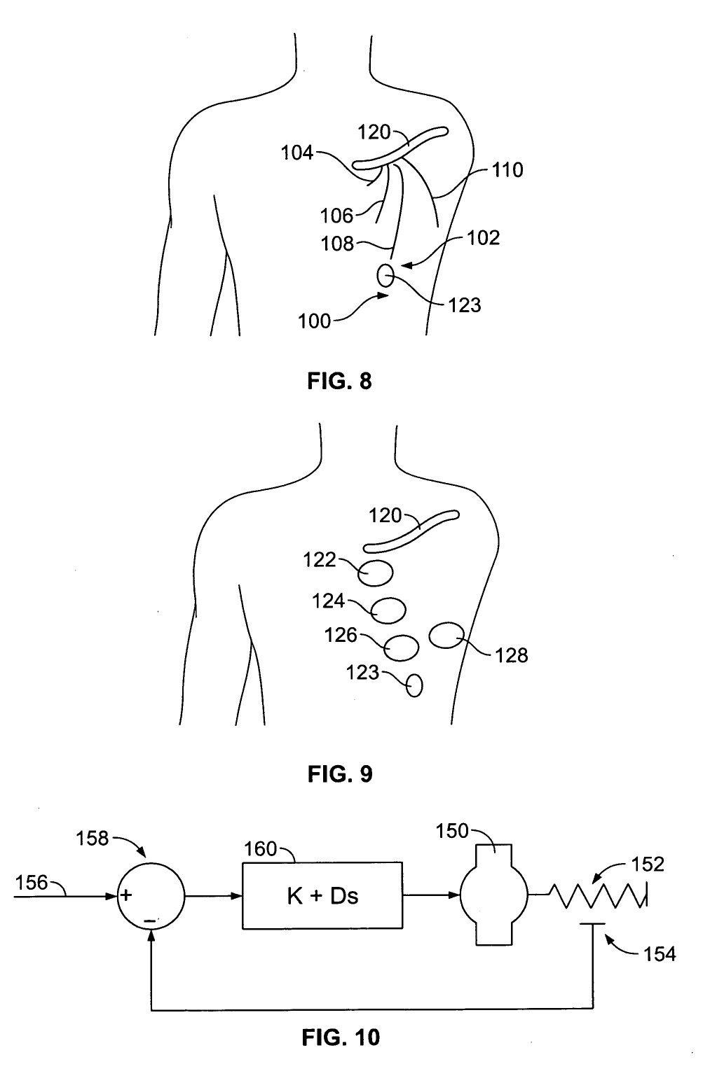

Using targeted reinnervation to transfer nerves from a lost limb to denervated pectoralis major and minor muscles 100 and 102 as shown in FIG. 8, sensation of the lost limb may be achieved on the chest of a subject. Four independently controlled nerve-muscle units below clavicle 120 and above nipple 123 can be created by surgically anatomizing residual brachial plexus nerves 104, 106, 108, and 110 (musculocutaneous nerve, median nerve; radial nerve and ulnar nerve respectively) to dissected and divided aspects of the pectoralis major and minor muscles 100, 102. Sensory reinnervation will occur on the chest in areas where the skin is denervated (the skin nerves are cut).

As a result of the surgery, the subject will perceive touch that appears to originate from the prosthetic limb when pressure is applied to the chest at points 122-128, of FIG. 9. A distinct representation will be acknowledged: pushing in one area will elicit perceived pressure or transfer sensation corresponding to one area of the limb replaced by the prosthesis whereas pushing in the other areas will elicit transfer sensation corresponding to different identifiable areas of the limb replaced by the prosthesis.

A like result can be achieved by denervating an available area of the skin, and reinnervating the denervated skin directly.

Actuator Selection

In order to achieve physiologically appropriate force feedback, an accurate force must be exerted against the chest. Because the chest moves with breathing, this matter becomes more complicated: an accurate force is desired, but the force must track the changing position of the chest. A linear backdrivable Series Elastic Actuator (SEA) can be used to decouple the inertia of the actuator from the force of the actuator. SEA are force controllable actuators with low impedance, high fidelity, and moderate bandwidth. They can be used to convert the accurate position control of traditional DC motors to accurate force control through the use of a spring as shown in FIG. 10. They have several advantageous properties, including reliable force output, simplicity, robustness of design, and the use of traditional robotic actuators. Most importantly, the compliant spring used in the device decouples inertia from force, especially at higher frequencies where inertia dominates the response.

Motor 150 thus generates an accurate position. This position is fed through a compression spring 152 that converts the accurate position into an accurate force. A linear potentiometer 154 that measures the compression and converts it into a force reading, which is then compared to the desired force 156 by a comparator 158. The error between the two signals is sent to a control block 160. The control block multiplies the error signal by a gain (K) and the derivative of the error by another gain (D) and sends this signal to the motor 150 to correct the output force. The forces can be nonlinearly scaled to provide increased resolution for low magnitude forces and/or decreased resolution for high magnitude forces.

### RESULTS

Testing of a system as described above with a linear conversion from hand force to chest force and a nonlinear conversion from hand force to chest force will produce increased resolution of low forces helping the subject discriminate low forces more effectively.

The subject's breathing will not disrupt his perception of force. When the subject is given various scale weights, he should be able to subjectively assess the inertia of the actuator.

2. An area of reinnervated skin was tested by applying carefully measured force to the skin. The patient was found to have a light touch threshold (first perception of being touched) of 2 g/mm², which is very close to normal values for natural limbs. Transfer sensation in terms of sensitivity to graded higher and lower pressures was found to be about one-half of the normal value for natural limbs. The patient had normal hot and cold perception and was able to distinguish between sharper and duller stimuli.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What we claim is:

1. A method for providing sensory feedback from an external prosthesis to an amputee by performing the steps of:
reinnervating a portion of the skin of the amputee with nerves that formerly provided sensory feedback from a lost limb replaced by the prosthesis;
providing a sensory indication from the prosthesis; and
using the sensory indication to produce a corresponding sensory condition adjacent the surface of the reinnervated skin to provide the sensory feedback to the amputee.

2. The method of claim 1 in which the skin is reinnervated by residual nerves of the lost limb.

3. The method of claim 2 in which the nerves to the skin portion are cut to facilitate the nerve-reinnervation thereof.

4. The method of claim 1 in which the reinnervated skin is probed to identify the portions of the reinnervated skin that correspond to sensory indications from identifiable locations on the lost limb.

5. The method of claim 4 in which sensory conditions are produced adjacent to one or more identifiable portions of reinnervated skin that correspond to sensory indications from identifiable locations on the lost limb.

6. The method of claim 1 in which the prosthesis is for arm or leg amputations.

7. The method of claim 1 in which the sensory indication and sensory condition are pressure.

8. The method of claim 1 in which the sensory indication and sensory condition are temperature.

9. The method of claim 1 in which the sensory indication is texture of a surface and the indication of texture is produced by passing a pressure sensor on the prosthesis along the surface.

10. The method of claim 1 in which the sensory indication is of a sharp/dull or an edge feature.

11. The method of claim 1 in which the sensory indication is of vibration.

12. The method of claim 1 in which the prosthesis is a motorized hand prosthesis with a force sensor in the prosthesis to provide the sensory indication of grip force.

13. The method of claim 12 in which the amputee controls the gripping force in response to the sensory condition.

14. The method of claim 1 in which a controller is used to receive the indication from the pressure sensor and to actuate an actuator to apply pressure to the reinnervated skin.

15. The method of claim 14 in which means are provided for monitoring and maintaining the desired level of pressure applied by the actuator.

16. The method of claim 1 in which the sensory condition adjacent to the surface of the reinnervated skin is scaled down from the sensory indication.

17. The method of claim 12 in which an actuator is provided to produce the sensory condition and is chosen from the group consisting of an elastic actuator, an electroactive polymer transducer, a linear solenoid having a plunger with an axial stroke, a motor controlled tactor, a pneumatic bladder, a piezo electric actuator, and a linear back-drivable series elastic actuator.

18. The method of claim 1 in which a Peltier device is provided to produce the sensory condition of temperature.

19. A system for providing sensory feedback to an amputee using an external prosthesis to enhance the functionality thereof comprising:
a portion of the skin of the amputee reinnervated with nerves that formerly provided sensory feedback from a lost limb replaced by the prosthesis;
a temperature or pressure sensor located on the prosthesis;
an actuator positioned adjacent the reinnervated skin to apply pressure, vibration, heating or cooling thereto; and
means coupling the sensor and actuator to produce a sensory condition at the actuator corresponding to the pressure, vibration or temperature sensed by the sensor.

20. The system of claim 19 in which the skin is reinnervated directly or by reinnervating adjacent muscle.

21. The system of claim 19 in which the reinnervated skin has identifiable portions innervated with nerves that correspond to sensory indications from identifiable locations on a lost limb.

22. The system of claim 21 in which selected sensors are provided corresponding to the sensory indications from identifiable locations on the lost limb and transducers coupled to the selected sensors are positioned adjacent the appropriate portions of the reinnervated skin.

23. The system of claim 19 in which more than one sensors and a corresponding number of actuators are used.

24. The system of claim 19 including means for scaling down the sensory condition produced by the actuator.

25. The system of claim 19 including means for scaling down in a non-linear fashion the sensory condition produced by the actuator.

26. The system of claim 19 in which the pressure sensor is chosen from the group consisting of an elastic actuator, an electroactive polymer transducer, a linear solenoid having a plunger with an axial stroke, a motor controlled plunger, a pneumatic bladder, and a piezoelectric actuator.

27. The system of claim 19 in which the temperature sensor is a thermistor.

28. The system of claim 19 in which the prosthesis is an arm, hand, leg or foot prosthesis.

29. The system of claim 19 in which the prosthesis is a hand prosthesis with motor-driven fingers and a pressure sensor is located at the tip of at least one finger of the prosthesis to provide the sensory indication.

30. The system of claim 19 in which the prosthesis is a foot prosthesis with at least one pressure sensor located on the bottom of the foot.

31. The system of claim 30 in which a leg prosthesis is attached to the foot prosthesis.

32. The system of claim 19 including a controller to receive an indication from a sensor corresponding to the level of pressure or temperature sensed and to actuate the actuator to exert a corresponding force on the appropriate portion of the reinnervated skin.

33. The system of claim 19 in which the sensor and actuator are coupled wirelessly.

34. The system of claim 19 including means for monitoring and maintaining the desired level of pressure, vibration or temperature applied by the actuator.
